(19) United States
(12) Patent Application Publication
(10) Pub. No.: US 2007/0198096 A1
(43) Pub. Date: Aug. 23, 2007

(54) Prosthetic joint

(76) Inventor: Christopher John Howard Wort, Oxfordshire (GB)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/572,288

(22) PCT Filed: Sep. 15, 2004
(86) PCT No.: PCT/IB04/02999

§ 371 Date: Jan. 16, 2007

Related U.S. Application Data
(60) Provisional application No. 60/506,778, filed on Sep. 30, 2003.

(30) Foreign Application Priority Data
Sep. 19, 2003 (GB) 0322020.9

(51) Int. Cl. A61F 2/30

(52) U.S. Cl. 623/23.42; 623/23.51

(57) ABSTRACT

A prosthetic joint ball is receivable in a prosthetic joint socket to form a prosthetic joint. The ball is generally spherical and is mounted on a stem. The outer rounded surface of the ball is provided with a layer of CVD diamond, prepared by mechanical polishing to present a surface with an RA of 20 nm or less. The ball is formed of a silicon carbide or similar substrate and includes a rounded recess. Mounted in the recess is a metal insert formed of a cobalt-chromium alloy or titanium alloy. The metal insert includes a socket shaped and sized to receive, in use, the tapered end of the stem.

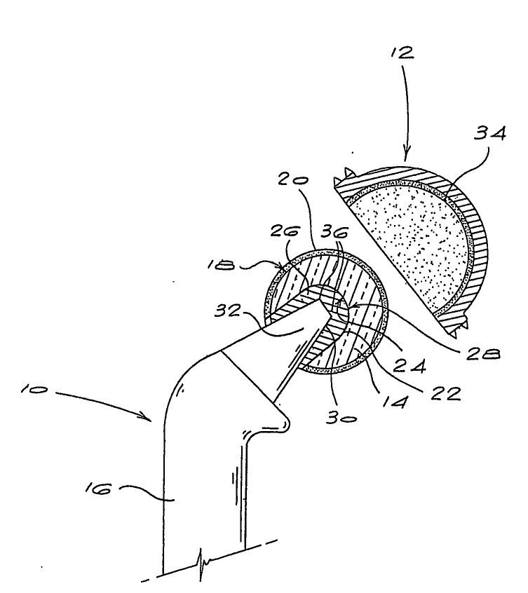

### BACKGROUND OF THE INVENTION

THIS invention relates to an orthopaedic implant, in particular a prosthetic joint component.

Orthopaedic implants or prosthetic joints are used extensively in the replacement of damaged or destroyed human joints, including hip and knee joints, for example. Prosthetic joints suffer from limited life spans, typically of the order of 15 years or less, whilst the market requirement for prosthetic joints is that they are viable for much greater periods.

Current prosthetic joints typically consist of a generally spherical ball formed of cobalt-chromium or titanium alloy, which is attached via a “stem” or joint hinge to a so-called long bone, and a hemispherical cup or socket that replaces the acetabular cup, and which is lined with ultra-high molecular weight polyethylene (UHMWPE).

Such ball and socket arrangements, however, invariably result in frictional wear of the spherical ball and the UHMWPE lining, releasing very fine particles. The fine particles cause histiocytic reactions in the body in an attempt to eliminate these particles. As a consequence, osteolysis takes place as agents, which are released as a result of the histiocytic reactions, attack the neighbouring bone, causing joint loosening and ultimately joint failure. Various attempts have been made to address this and other problems associated with prosthetic joints.

For instance, attempts have been made to use metal balls and sockets made from biocompatible materials. However, the problems of wear debris being formed as a result of friction still occur, with the same consequences as previously mentioned.

Another alternative is to form the ball of a biocompatible ceramic and to coat the ceramic with a biocompatible non-wear material, such as diamond. Whilst this addresses the problems associated with wear debris, potential for prosthetic joint failure due to other causes still exists.

In practice, the ceramic ball is attached to a stem having a tapered profile end, which is the industry standard stem used in joint replacements. A problem with ceramics is that although mechanically strong under compression, they generally have a low fracture strength under tension. Accordingly, the stress concentrations at the corners of the tapered stem in the ceramic ball are prone to cause fractures in the ceramic and thus premature joint failure.

### SUMMARY OF THE INVENTION

According to an aspect of the invention, a prosthetic joint component comprises:
a generally spherical substrate presenting a bearing or wear-resistant surface for the component, and having a recess extending into the substrate; and
a metal insert secured in the recess, the metal insert having a socket sized and shaped to receive an end of a stem or joint hinge.

The substrate preferably includes a wear resistant layer or coating of material on an outer surface thereof, which wear resistant layer or coating defines the bearing or wear resistant surface presented by the substrate.

The substrate and/or, where appropriate, the wear resistant layer or coating can be readily shaped to present the desired bearing or wear-resistant surface. The substrate is refractory and mechanically tough to provide mechanical strength and support for the wear-resistant layer or surface. The wear resistant surface must in addition be smooth, typically polished to an R_{A} of 20 nm or less.

The substrate is preferably a ceramic substrate.

The ceramic substrate is typically Si₃N₄, SiC, SiALON, Al₂O₃ or ZrO, with the optional addition of impurities to control properties such as grain size.

In a preferred embodiment of the invention a layer of wear resistant material is bonded to the substrate surface by any means known in the art. Possible wear resistant materials include other ceramic materials, diamond-like carbon (DLC) and the like as well as diamond.

The wear-resistant material bonded to the surface of the substrate is preferably a layer of diamond, which may be single crystal or polycrystalline, polycrystalline CVD diamond being particularly preferred.

The wear resistant layer may be bonded as a discrete object to the substrate by any means known in the art, or it may be applied or directly synthesised onto the substrate using any of the deposition techniques reported in the art. In the case where the coating is CVD diamond, appropriate synthesis techniques include microwave plasma assisted CVD and hot filament CVD as are well reported in the literature.

Where used, the CVD diamond layer is typically 100 to 500 μm thick. In final form it must present a smooth surface with an R_{A} of 20 nm or less, so that techniques to control or refine the grain size of the polycrystalline diamond may be used, and the final layer may be polished using any of the mechanical or chemical processing methods known in the art.

Where the substrate is to be provided with an additional wear resistant layer by coating or bonding, the surface of the substrate may be optimised for use with the coating. For example, to aid adhesion the substrate surface may be scored or roughened, or an interlayer or bonding material used, or in the case of using a wear resistant coating with an inherently low surface roughness, such as an amorphous material, the surface of the substrate may be polished so that the final surface of the coating needs minimal or no further processing in order to present a suitably smooth surface.

The metal insert may be secured in the recess of the substrate by brazing, for example using TiCuAg (often referred to as TiCuSil), or diffusion bonding, for example using Ti or Au. In a preferred embodiment the metal insert is selected to have a thermal expansion coefficient close to that of the substrate.

The recess of the substrate and the metal insert preferably comprise respective complimentary non-planar inner and outer surfaces, which includes the configuration where surfaces are slightly modified from being perfectly mechanically complementary in order to accommodate or assist in the method of bonding. The complementary non-planar surfaces of the recess and metal insert are preferably rounded.

The socket of the metal insert is preferably sized and shaped to receive the tapered end of a stem or metal hinge.

The metal insert is preferably made of a cobalt-chromium alloy, titanium, zirconium, tungsten or stainless steel, although it may be any appropriate tough metal or metal alloy that is biocompatible.

The invention extends to a prosthetic joint comprising a prosthetic joint component as described above and a socket component sized and shaped to receive the wear-resistant surface of the prosthetic joint component.

The socket component is preferably also coated on an inner surface thereof with a wear-resistant material as described above, in particular CVD diamond.

### BRIEF DESCRIPTION OF THE DRAWING

The invention will now be described in more detail, by way of example only, with reference to the accompanying drawing, which is a sectional side view of a prosthetic joint incorporating a prosthetic joint component of the invention.

### DESCRIPTION OF PREFERRED EMBODIMENTS

Referring to the accompanying drawing, a prosthetic joint ball component 10 of the invention is receivable in a prosthetic joint socket component 12 to form a prosthetic joint. The ball component 10 comprises a generally spherical ball 14 mounted on a stem 16. The outer rounded surface 18 of the ball 14, in this case, is provided with a layer 20 of CVD diamond, prepared by mechanical polishing to present a surface with an R_{A} of 20 nm or less. The ball 14 is formed of a silicon carbide or similar substrate and includes a recess 22 having a rounded surface 24. Mounted in the recess 22 is a metal insert 26 formed of a cobalt-chromium alloy or titanium alloy, and having a rounded outer surface 28 complementary to the surface 24 of the recess 22. The metal insert 26 includes a socket 30 which is shaped and sized to receive, in use, the tapered end 32 of the stem 16.

The metal insert 26 is bonded in the recess 22 by brazing using, for example, TiCuAg, or diffusion bonding using, for example, Ti or Au.

The socket component 12 is also lined with a layer of CVD diamond 34, prepared by mechanical polishing to present a surface with an R_{A} of 20 nm or less, to reduce friction between the ball 10 and the socket 12, reduce wear and thereby reduce the risk of wear debris forming.

By providing a metal insert 26, the high stress concentrations at the corners 36 are applied to a tough metal, which is not prone to cracking or fracturing as would be the case if the stem was bonded directly to the ceramic substrate. Further, the bonding between the metal insert 26 and the ceramic is a low stress design, i.e. there are no sharp stress concentrators and the load is spread over a rounded surface, thus drastically reducing the tendency of the substrate to fracture. By utilising a polished diamond layer on the ceramic substrate, a prosthetic joint component is provided which is biocompatible, and has lower friction and lower wear resulting in debris free use thereof in a prosthetic joint.

1-20. (canceled)

21. A prosthetic joint component comprising:
a generally spherical substrate presenting a bearing or wear-resistant surface for the joint component and having a recess extending into the substrate, the recess defining a non-planar inner surface; and
a metal insert having a non-planar outer surface complementary to that of the recess inner surface, the metal insert being secured in the recess and having a socket sized and shaped to receive an end of a stem or joint hinge.

22. A prosthetic joint component according to claim 21, wherein the complementary non-planar surfaces of the recess and metal insert are rounded.

23. A prosthetic joint component according to claim 21, wherein the substrate is a ceramic substrate.

24. A prosthetic joint component according to claim 23, wherein the ceramic substrate is selected from the group of Si₃N₄, SiC, SiALON, Al₂O₃, and ZrO, with optional addition of impurities to control properties thereof.

25. A prosthetic joint component according to claim 21, wherein the metal insert is secured in the recess of the substrate by brazing or diffusion bonding.

26. A prosthetic joint component according to claim 21, wherein the metal insert is selected to have a thermal expansion coefficient close to that of the substrate.

27. A prosthetic joint component according to claim 21, wherein the metal insert is made of a cobalt-chromium alloy, titanium, zirconium, tungsten, stainless steel, or other tough metal or metal alloy that is biocompatible.

28. A prosthetic joint component according to claim 21, wherein the substrate comprises a wear-resistant layer or coating on an outer surface thereof, which wear-resistant layer or coating defines the bearing or wear-resistant surface presented by the substrate.

29. A prosthetic joint component according to claim 28, wherein the wear-resistant layer is bonded to the substrate surface.

30. A prosthetic joint component according to claim 28, wherein the wear-resistant layer is formed from a ceramic material, diamond-like carbon (DLC) material, or diamond.

31. A prosthetic joint component according to claim 30, wherein the wear-resistant layer is formed from polycrystalline CVD diamond.

32. A prosthetic joint component according to claim 28, wherein the wear-resistant layer is bonded as a discrete component to the substrate or is applied or directly synthesised onto the substrate.

33. A prosthetic joint component according to claim 28, wherein the wear-resistant layer is a CVD diamond layer that is 100 to 500:m thick.

34. A prosthetic joint component according to claim 21, wherein the substrate and/or the wear-resistant layer or coating is shaped to present the bearing or wear-resistant surface.

35. A prosthetic joint component according to claim 21, wherein the bearing or wear-resistant surface is smooth.

36. A prosthetic joint component according to claim 35, wherein the bearing or wear-resistant surface is polished to an R_{A} of 20 nm or less.

37. A prosthetic joint component according to claim 21, wherein the socket of the metal insert is sized and shaped to receive a tapered end of a stem or metal hinge.

38. A prosthetic joint comprising a prosthetic joint component according to claim 21 and a socket component sized and shaped to engage the wear-resistant surface of the prosthetic joint component.

39. A prosthetic joint according to claim 38, wherein the socket component is coated on an inner surface thereof with a wear-resistant material.
